(19) United States
(12) Patent Application Publication
(10) Pub. No.: US 2026/0103528 A1
(43) Pub. Date: Apr. 16, 2026

(54) NANOBODIES THAT BIND TO GPRC5D AND USES THEREOF

(71) Applicant: BIOFRONT THERAPEUTICS (BEIJING) CO., LTD., Beijing (CN)

(72) Inventors: Daopeng YUAN, Beijing (CN); Chunyan WU, Beijing (CN); Hui ZHANG, Beijing (CN); Jie ZHU, Beijing (CN)

(73) Assignee: BIOFRONT THERAPEUTICS (BEIJING) CO., LTD., Beijing (CN)

(21) Appl. No.: 19/333,636

(22) Filed: Sep. 19, 2025

Related U.S. Application Data
(63) Continuation of application No. PCT/CN2023/082991, filed on Mar. 22, 2023.

(51) Int. Cl. C07K 16/28

(52) U.S. Cl. CPC C07K 16/2866; C07K 2317/52; C07K 2317/569; C07K 2317/92

(57) ABSTRACT

Provided herein is a nanobody that binds to GPRC5D, an antibody, an immunoconjugate, and a pharmaceutical composition that include the nanobody, an isolated nucleic acid encoding the nanobody, a host cell expressing the nanobody, and relevant methods and applications.

### CROSS-REFERENCE TO THE RELATED APPLICATIONS

This application is a Continuation of International Application No. PCT/CN2023/082991, filed on Mar. 22, 2023, the contents of which are hereby incorporated by reference.

### SEQUENCE LISTING

The instant application contains a Sequence Listing which is submitted electronically in XML format and is hereby incorporated by reference in its entirety. The XML copy, created on Aug. 27, 2025, is named “2025 Aug. 27-Sequence Listing-20752-0003US00,” and is 26,968 bytes in size.

### TECHNICAL FIELD

The present disclosure relates to the technical field of biomedicine, and in particular, to an nanobodies that bind to GPRC5D and uses thereof.

### BACKGROUND

Multiple myeloma (MM) is a malignant proliferative disease of plasma cells. Clonal plasma cells in the bone marrow of patients with MM proliferate abnormally and secrete monoclonal immunoglobulins or fragments thereof, resulting in myeloma-related organ or tissue impairment (ROTI), such as hypercalcemia, renal insufficiency, anemia, and bone pain. MM accounts for about 10% of hematological malignancies and is the second most common malignancy of the hematological system in most countries in the world. Currently, MM is still considered an incurable disease. For patients who are relatively young (e.g., younger than 65 years old) and suitable for hematopoietic stem cell transplantation, autologous stem cell transplantation combined with drug therapy can effectively prolong the survival of these patients. For patients who are relatively old (e.g., over 65 years old) and/or not suitable for transplantation, more effective drug therapy regimens are urgently needed to prolong survival and improve quality of life. In recent years, immunomodulators (IMiDs), proteasome inhibitors (Pls), antibody drugs and the derivatives thereof, small molecule targeted therapeutic drugs, etc. have shown good prospects in the treatment of MM. However, after the disease relapses or drug resistance develops, the effect of the drug therapy regimen previously taken by MM patients inevitably diminishes gradually. Therefore, the treatment of MM faces the problem of limited available drugs and urgent research and development of new drugs.

G protein-coupled receptor class C group 5 member D (GPRC5D) is a G protein-coupled orphan receptor, which is expressed in normal tissues to a limited extent and a high level in MM cells. GPRC5D has been reported to serve as a potential target for the treatment of MM.

Compared with traditional antibodies, nanobodies have the advantages of having a small molecular weight, more easily penetrating tissues, and reaching tumor areas. Nanobodies can achieve higher effective therapeutic concentrations in a wider tumor area to improve therapeutic efficacy. At the same time, the lack of light chains in nanobodies helps to avoid light chain mismatches of bispecific/multispecific antibodies. More importantly, nanobodies are hydrophilic and cross-linking and a plurality of nanobodies can be linked to form desired types of bispecific/multispecific antibodies. Therefore, the development of nanobodies and the derivative drugs thereof for GPRC5D has a positive impact on promoting the drug therapy of MM.

### SUMMARY

According to a first aspect of the present disclosure, a nanobody that binds specifically to G protein-coupled receptor, class C, group 5, member D (GPRC5D) is provided. The nanobody may include a combination of three complementary determining regions (CDRs). The combination may include a CDR1 having an amino acid sequence as set forth in SEQ ID NO: 13, a CDR2 having an amino acid sequence as set forth in SEQ ID NO: 14, and a CDR3 having an amino acid sequence as set forth in SEQ ID NO: 15.

In some embodiments, the nanobody may include a VHH chain having an amino acid sequence as set forth in SEQ ID NO: 3.

In some embodiments, the nanobody may be used as a medicament.

In some embodiments, the nanobody may be used in treating a disorder or disease related to GPRC5D. Further, the nanobody may be used in treating multiple myeloma (MM).

According to a second aspect of the present disclosure, a nanobody that binds specifically to G protein-coupled receptor, class C, group 5, member D (GPRC5D) is provided. The nanobody may include a combination of three complementary determining regions (CDRs). The combination may include a CDR1 having an amino acid sequence as set forth in SEQ ID NO: 16, a CDR2 having an amino acid sequence as set forth in SEQ ID NO: 17, and a CDR3 having an amino acid sequence as set forth in SEQ ID NO: 18.

In some embodiments, the nanobody may include a VHH chain having an amino acid sequence as set forth in SEQ ID NO: 4.

In some embodiments, the nanobody may be used as a medicament.

In some embodiments, the nanobody may be used in treating a disorder or disease related to GPRC5D. Further, the nanobody may be used in treating multiple myeloma (MM).

According to a third aspect of the present disclosure, a nanobody that binds specifically to G protein-coupled receptor, class C, group 5, member D (GPRC5D) is provided. The nanobody may include a combination of three complementary determining regions (CDRs). The combination may include a CDR1 having an amino acid sequence as set forth in SEQ ID NO: 19, a CDR2 having an amino acid sequence as set forth in SEQ ID NO: 20, and a CDR3 having an amino acid sequence as set forth in SEQ ID NO: 21.

In some embodiments, the nanobody may include a VHH chain having an amino acid sequence as set forth in SEQ ID NO: 5.

In some embodiments, the nanobody may be used as a medicament.

In some embodiments, the nanobody may be used in treating a disorder or disease related to GPRC5D. Further, the nanobody may be used in treating multiple myeloma (MM).

According to a fourth aspect of the present disclosure, a nanobody that binds specifically to G protein-coupled receptor, class C, group 5, member D (GPRC5D) is provided. The nanobody may include a combination of three complementary determining regions (CDRs). The combination may include a CDR1 having an amino acid sequence as set forth in SEQ ID NO: 22, a CDR2 having an amino acid sequence as set forth in SEQ ID NO: 23, and a CDR3 having an amino acid sequence as set forth in SEQ ID NO: 24.

In some embodiments, the nanobody may include a VHH chain having an amino acid sequence as set forth in SEQ ID NO: 6.

In some embodiments, the nanobody may be used as a medicament.

In some embodiments, the nanobody may be used in treating a disorder or disease related to GPRC5D. Further, the nanobody may be used in treating multiple myeloma (MM).

According to a fifth aspect of the present disclosure, a nanobody that binds specifically to G protein-coupled receptor, class C, group 5, member D (GPRC5D) is provided. The nanobody may include a combination of three complementary determining regions (CDRs). The combination may include a CDR1 having an amino acid sequence as set forth in SEQ ID NO: 25, a CDR2 having an amino acid sequence as set forth in SEQ ID NO: 26, and a CDR3 having an amino acid sequence as set forth in SEQ ID NO: 27.

In some embodiments, the nanobody may include a VHH chain having an amino acid sequence as set forth in SEQ ID NO: 7.

In some embodiments, the nanobody may be used as a medicament.

In some embodiments, the nanobody may be used in treating a disorder or disease related to GPRC5D. Further, the nanobody may be used in treating multiple myeloma (MM).

According to a sixth aspect of the present disclosure, an anti-GPRC5D antibody is provided. The antibody may include the nanobody.

In some embodiments, the antibody may be bispecific, bind to GPRC5D, and further bind to a second antigen.

In some embodiments, the antibody may be used as a medicament.

In some embodiments, the antibody may be used in treating a disorder or disease related to GPRC5D. Further, the antibody may be used in treating multiple myeloma (MM).

According to a seventh aspect of the present disclosure, an immunoconjugate is provided. The immunoconjugate may include the nanobody.

In some embodiments, the immunoconjugate may include an antibody that includes the nanobody.

In some embodiments, the antibody may be conjugated to a cytotoxic agent, a radioisotope, a drug compound, or a peptide.

In some embodiments, the immunoconjugate may be used as a medicament.

In some embodiments, the immunoconjugate may be used in treating a disorder or disease related to GPRC5D. Further, the immunoconjugate may be used in treating multiple myeloma (MM).

According to an eighth aspect of the present disclosure, an isolated nucleic acid is provided. The isolated nucleic acid may encode the nanobody.

In some embodiments, the isolated nucleic acid may be used as a medicament.

In some embodiments, the isolated nucleic acid may be used in treating a disorder or disease related to GPRC5D. Further, the isolated nucleic acid may be used in treating multiple myeloma (MM).

According to a ninth aspect of the present disclosure, a recombinant eukaryotic or prokaryotic host cell is provided. The host cell may produce the nanobody.

According to a tenth aspect of the present disclosure, a pharmaceutical composition is provided. The pharmaceutical composition may include the nanobody.

In some embodiments, the pharmaceutical composition may be used as a medicament.

In some embodiments, the pharmaceutical composition may be used in treating a disorder or disease related to GPRC5D. Further, the pharmaceutical composition may be used in treating multiple myeloma (MM).

According to an eleventh aspect of the present disclosure, a pharmaceutical composition is provided. The pharmaceutical composition may include the antibody, the immunoconjugate, or the isolated nucleic acid.

In some embodiments, the pharmaceutical composition may be used as a medicament.

In some embodiments, the pharmaceutical composition may be used in treating a disorder or disease related to GPRC5D. Further, the pharmaceutical composition may be used in treating multiple myeloma (MM).

According to a twelfth aspect of the present disclosure, a method for treating a patient suffering from a disease related to GPRC5D is provided. The method may include administering a pharmacologically effective amount of the nanobody, the antibody, the immunoconjugate, the isolated nucleic acid, or the pharmaceutical composition to the patient.

In some embodiments, the disease may be multiple myeloma (MM).

According to a thirteenth aspect of the present disclosure, a method for detecting the presence of GPRC5D in a sample is provided. The method may include: contacting the sample with the nanobody under conditions that allow for formation of a complex between the nanobody and GPRC5D; and determining whether the complex has been formed.

According to a fourteenth aspect of the present disclosure, a method for producing an anti-GPRC5D nanobody is provided. The method may include: (i) culturing a host cell that expresses a nucleic acid encoding the nanobody, and (ii) purifying the anti-GPRC5D nanobody from a culture medium of the host cell.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, wherein:

FIG. 1 respectively show schematic diagrams of an anti-GPRC5D nanobody (part A) and a recombinant antibody (part B) constructed by fusion of the anti-GPRC5D nanobody and an IgG-Fc according to some embodiments of the present disclosure;

FIGS. 2(a)-2(c) show a purity identification result of a candidate recombinant antibody 1-F7-FC-fusion constructed by fusion of a candidate nanobody 1-F7 and the IgG-Fc according to some embodiments of the present disclosure; FIG. 2(a) is a high performance liquid chromatography-size exclusion chromatography (HPLC-SEC) graph of the candidate recombinant antibody (a detection wavelength of 214 nm); FIG. 2(b) is a HPLC-SEC graph of the candidate recombinant antibody (a detection wavelength of 280 nm); FIG. 2(c) is a sodium dodecyl sulfate-polyacrylamide gel electrophoresis (SDS-PAGE) graph of the candidate recombinant antibody under non-reducing (NR) and reducing (R) conditions;

FIGS. 3(a)-3(c) show a purity identification result of a candidate recombinant antibody 1-E8-FC-fusion constructed by fusion of a candidate nanobody 1-E8 and the IgG-Fc according to some embodiments of the present disclosure; FIG. 3(a) is a HPLC-SEC graph of the candidate recombinant antibody (a detection wavelength of 214 nm); FIG. 3(b) is a HPLC-SEC graph of the candidate recombinant antibody (a detection wavelength of 280 nm); FIG. 3(c) is a SDS-PAGE graph of the candidate recombinant antibody under non-reducing (NR) and reducing (R) conditions;

FIGS. 4(a)-4(c) show a purity identification result of a candidate recombinant antibody 1-D9-FC-fusion constructed by fusion of a candidate nanobody 1-D9 and the IgG-Fc according to some embodiments of the present disclosure; FIG. 4(a) is a HPLC-SEC graph of the candidate recombinant antibody (a detection wavelength of 214 nm); FIG. 4(b) is a HPLC-SEC graph of the candidate recombinant antibody (a detection wavelength of 280 nm); FIG. 4(c) is a SDS-PAGE graph of the candidate recombinant antibody under non-reducing (NR) and reducing (R) conditions;

FIGS. 5(a)-5(c) show a purity identification result of a candidate recombinant antibody 1-C12-FC-fusion constructed by fusion of a candidate nanobody 1-C12 and the IgG-Fc according to some embodiments of the present disclosure; FIG. 5(a) is a HPLC-SEC graph of the candidate recombinant antibody (a detection wavelength of 214 nm); FIG. 5(b) is a HPLC-SEC graph of the candidate recombinant antibody (a detection wavelength of 280 nm); FIG. 5(c) is a SDS-PAGE graph of the candidate recombinant antibody under non-reducing (NR) and reducing (R) conditions;

FIGS. 6(a)-6(c) show a purity identification result of a candidate recombinant antibody 1-G7-FC-fusion constructed by fusion of a candidate nanobody 1-G7 and the IgG-Fc according to some embodiments of the present disclosure; FIG. 6(a) is a HPLC-SEC graph of the candidate recombinant antibody (a detection wavelength of 214 nm); FIG. 6(b) is a HPLC-SEC graph of the candidate recombinant antibody (a detection wavelength of 280 nm); FIG. 6(c) is a SDS-PAGE graph of the candidate recombinant antibody under non-reducing (NR) and reducing (R) conditions;

FIG. 7 shows a graph illustrating binding abilities of the phage-displayed candidate nanobodies to a purified GPRC5D protein detected by an enzyme-linked immunosorbent assay (ELISA) according to some embodiments of the present disclosure and the ordinate represents an absorbance value at 450 nm;

FIG. 8 shows a graph illustrating binding abilities of the phage-displayed candidate nanobodies to a CHOK1 cell transfected to express human GPRC5D (CHOK1-GPRC5D) detected by the ELISA according to some embodiments of the present disclosure, the control group is untransfected CHOK1 cells (CHOK1), and the ordinate represents an absorbance value at 450 nm;

FIG. 9 shows a graph illustrating binding abilities of recombinant antibodies constructed by fusion of candidate nanobodies and Fc fragments to a cell expressing human GPRC5D detected by the ELISA according to some embodiments of the present disclosure, the ordinate represents an absorbance value at 450 nm, the cell line expressing human GPRC5D includes a CHOK1-C5D, an MM1R, and an H929, and the control group is untransfected CHOK1 cells (CHOK1);

FIGS. 10(a)-10(e) show histograms illustrating binding abilities of the phage-displayed candidate nanobodies detected by a fluorescence-activated cell sorting (FACS) to a CHOK1-GPRC5D cell overexpressing human GPRC5D according to some embodiments of the present disclosure, the abscissa represents a relative intensity of a fluorescent signal, and the ordinate represents a relative count of cells/particles;

FIGS. 11(a)-11(e) show histograms illustrating binding abilities of the phage-displayed candidate nanobodies to an H929 cell expressing human GPRC5D detected by the FACS according to some embodiments of the present disclosure, the abscissa represents a relative intensity of a fluorescent signal, and the ordinate represents a relative count of cells/particles; and

FIGS. 12(a)-12(e) show histograms illustrating binding abilities of the phage-displayed candidate nanobodies to an MM1R cell expressing human GPRC5D detected by the FACS according to some embodiments of the present disclosure, the abscissa represents a relative intensity of a fluorescent signal, and the ordinate represents a relative count of cells/particles.

### DETAILED DESCRIPTION

In order to more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that although the terms “first,” “second,” “third,” etc. may be used herein to describe various elements, these elements should not be limited by these terms. These terms are merely used to distinguish one element from another. For example, a first product may be referred to as a second product. Similarly, a second product may be referred to as a first product, without departing from the scope of exemplary embodiments of the present disclosure.

As used in the disclosure and the appended claims, the singular forms “a,” “an,” and “the” include plural referents unless the content clearly dictates otherwise; the plural forms may be intended to include singular forms as well. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

Unless defined otherwise, all technical and scientific terms used herein have the same meaning as those commonly understood by those skilled in the art to which the present disclosure belongs.

As used herein, the term “antibody” or “Ab” refers to a molecule including at least one immunoglobulin heavy chain (HC) and at least one immunoglobulin light chain (LC). Each heavy chain may include a heavy chain variable region (VH) and a heavy chain constant region (CH). The heavy chain variable region may have 3 complementary determining regions (CDRs) and 4 framework regions (FRs). Each light chain may include a light chain variable region (VL) and a light chain constant region (CL). The light chain variable region may have 3 complementary determining regions and 4 framework regions. According to the different antigenicity of VH, the antibody may be divided into IgM, IgG, IgA, IgD, and IgE. In some embodiments, the antibody may include, but is not limited to, a monoclonal antibody, a bispecific antibody, a multispecific antibody, a dimeric antibody, a trimeric antibody, a multimeric antibody, etc.

The term “CDR” or “complementary determining region” refers to a region where the antibody specifically recognizes an antigen.

The term “Fc,” “Fc fragment,” or “fragment crystallizable region” refers to a terminal region of an antibody that is capable of binding to various cell surface receptors (e.g., Fc receptors) and complement proteins.

The term “Nanobody,” “VHH,” or “single domain antibody” refers to an antibody consisting of a single monomeric variable antibody domain, which is a smallest antigen-binding fragment with full functionality.

The term “peptide” refers to a compound formed by dehydration and condensation of at least two amino acid molecules.

The term “purified” refers that a nucleic acid or a peptide is present in the substantial absence of other biological macromolecules.

The term “isolated” refers that a nucleic acid or a peptide is separated from one or more other components (e.g., other genomic nucleic acid sequences, proteins, etc.) present in a source of the nucleic acid or the peptide.

The term “host cell” refers to a cell capable of introducing a foreign gene and stably maintaining the foreign gene. In some embodiments, host cells include prokaryotic and eukaryotic cells. A non-limiting example of a host cell for expressing the nanobody may include, but is not limited to, an Escherichia coli, a yeast cell, an insect cell, a rodent cell, etc.

The term “identity” refers to a relationship between sequences of two or more peptide molecules or two or more nucleic acid molecules determined by aligning and comparing the sequences. “Percent identity” refers to comparing percentages of identical residues between amino acids or nucleotides in molecules and is calculated based on a size of a smallest molecule being compared.

The term “pharmacologically effective amount” refers to an effective dose for improving a disease symptom.

The term “administering” or “administration” refers to a process of administering a compound (e.g., a nanobody, an immunoconjugate, a pharmaceutical composition, etc.) for use as a medicament to a subject or patient. In some embodiments, an administration route may include, but is not limited to, an oral administration, an intramuscular injection, an intradermal injection, a subcutaneous injection, an intravenous injection, a pleural or intraperitoneal injection, an inhalation administration, an implantation administration, etc.

As discussed above, the treatment of MM faces the problem that available medicaments are limited and a new medicament needs to be developed urgently. GPRC5D may serve as a target for the treatment of MM. A new medicament (e.g., a nanobody targeting GPRC5D) may be designed for GPRC5D that can be used in the treatment of MM. It should be known that in terms of screening difficulty, the screening difficulty of a nanobody that relies on three CDRs-bound antigens is much higher than that of a traditional antibody that relies on six CDRs-bound antigens. It is difficult for those skilled in the art to obtain a nanobody with high affinity and high specificity. With in-depth and extensive research, the present disclosure points to nanobodies and recombinant antibodies thereof with high affinity and high specificity for GPRC5D protein and various types of cells expressing GPRC5D protein using phage display technology. Combined with features such as weak immunogenicity, easy purification, etc. of the nanobody, the findings can play an active role in the clinical research and the medicament production of MM, for example, in the development of various types of medicaments or therapies for the treatment of MM such as but not limited to CAR-T cells, nanobodies monoclonal antibodies, bispecific/multispecific antibodies, and antibody-drug conjugates.

Some embodiments of the present disclosure provide a nanobody that binds specifically to GPRC5D. The nanobody may include a combination of three CDRs and the combination may include a CDR1, a CDR2, and a CDR3.

In some embodiments, an amino acid sequence of the CDR1 may be as set forth in SEQ ID NO: 13. An amino acid sequence of the CDR2 may be as set forth in SEQ ID NO: 14. An amino acid sequence of the CDR3 may be as set forth in SEQ ID NO: 15.

In some embodiments, the nanobody may include a VHH chain having an amino acid sequence as set forth in SEQ ID NO: 3.

In some embodiments, the VHH chain may include a conservative substitution of less than or equal to 2 amino acids or less than or equal to 1 amino acid. As used herein, the term “conservative substitution” refers to an amino acid substitution that does not adversely affect or alter an essential property of a protein/peptide including the amino acid sequence. For example, the conservative substitution may be introduced by a standard technique (e.g., site-directed mutagenesis and polymerase chain reaction (PCR)-mediated mutagenesis) known in the art. A conservative amino acid substitution may include a substitution in which an amino acid residue is substituted by another amino acid residue having a similar side chain, such as a substitution of a physically or functionally similar residue (e.g., having a similar size, shape, charge, a chemical property including an ability to form a covalent bond or a hydrogen bond, etc.) to a corresponding amino acid residue. Families of the amino acid residues having similar side chains has been defined in the art. These families may include amino acids with basic side chains (e.g., lysine, arginine, and histidine), amino acids with acidic side chains (e.g., aspartic acid and glutamic acid), amino acids with uncharged polar side chains (e.g., glycine, asparagine, glutamine, serine, threonine, tyrosine, cysteine, and tryptophan), amino acids with non-polar side chains (e.g., alanine, valine, leucine, isoleucine, proline, phenylalanine, and methionine), amino acids with β-branched side chains (e.g., threonine, valine, and isoleucine), and amino acids with aromatic side chains (e.g., tyrosine, phenylalanine, tryptophan, and histidine). Accordingly, the corresponding amino acid residue may be preferably substituted by another amino acid residue from the same side chain family.

In some embodiments, the VHH chain may have an amino acid sequence with at least 90%, 92%, 94%, 96%, or 98% identity in the framework regions compared to the amino acid sequence as set forth in SEQ ID NO: 3. In some embodiments, the VHH chain may have an amino acid sequence having addition, deletion, and/or substitution of one or more amino acids in the framework regions compared to the amino acid sequence as set forth in SEQ ID NO: 3.

In some embodiments, the nanobody may be in an isolated state (or a purified state). For example, the nanobody may be produced by immunizing an animal (e.g., a camel) with a human GPRC5D antigen protein and biochemically isolated. In other embodiments, the nanobody may be recombinant. For example, the nucleic acid encoding the nanobody may be specifically amplified and isolated, the isolated nucleic acid encoding the nanobody may be cloned into suitable expression vectors, and the nanobody may be produced by transfecting suitable host cells with the expression vectors and biochemically isolated.

Some embodiments of the present disclosure also provide a nanobody that binds specifically to GPRC5D. The nanobody may include a combination of three CDRs and the combination may include a CDR1, a CDR2, and a CDR3.

In some embodiments, an amino acid sequence of the CDR1 may be set forth in SEQ ID NO: 16. An amino acid sequence of the CDR2 may be set forth in SEQ ID NO: 17. An amino acid sequence of the CDR3 may be set forth in SEQ ID NO: 18.

In some embodiments, the nanobody may include a VHH chain having an amino acid sequence as set forth in SEQ ID NO: 4.

In some embodiments, the VHH chain may include a conservative substitution of less than or equal to 2 amino acids or less than or equal to 1 amino acid.

In some embodiments, the VHH chain may have an amino acid sequence with at least 90%, 92%, 94%, 96%, or 98% identity in the framework regions compared to the amino acid sequence as set forth in SEQ ID NO: 4. In some embodiments, the VHH chain may have an amino acid sequence having addition, deletion, and/or substitution of one or more amino acids in the framework regions compared to the amino acid sequence as set forth in SEQ ID NO: 4.

In some embodiments, the nanobody may be in an isolated state. In other embodiments, the nanobody may be recombinant.

Some embodiments of the present disclosure also provide a nanobody that binds specifically to GPRC5D. The nanobody may include a combination of three CDRs and the combination may include a CDR1, a CDR2, and a CDR3.

In some embodiments, an amino acid sequence of the CDR1 may be as set forth in SEQ ID NO: 19. An amino acid sequence of the CDR2 may be as set forth in SEQ ID NO: 20. An amino acid sequence of the CDR3 may be as set forth in SEQ ID NO: 21.

In some embodiments, the nanobody may include a VHH chain having an amino acid sequence as set forth in SEQ ID NO: 5.

In some embodiments, the VHH chain may include a conservative substitution of less than or equal to 2 amino acids or less than or equal to 1 amino acid.

In some embodiments, the VHH chain may have an amino acid sequence with at least 90%, 92%, 94%, 96%, or 98% identity in the framework regions compared to the amino acid sequence as set forth in SEQ ID NO: 5. In some embodiments, the VHH chain may have an amino acid sequence having addition, deletion, and/or substitution of one or more amino acids in the framework regions compared to the amino acid sequence as set forth in SEQ ID NO: 5.

In some embodiments, the nanobody may be in an isolated. In other embodiments, the nanobody may be recombinant.

Some embodiments of the present disclosure also provide a nanobody that binds specifically to GPRC5D. The nanobody may include a combination of three CDRs and the combination may include a CDR1, a CDR2, and a CDR3.

In some embodiments, an amino acid sequence of the CDR1 may be as set forth in SEQ ID NO: 22. An amino acid sequence of the CDR2 may be as set forth in SEQ ID NO: 23. An amino acid sequence of the CDR3 may be as set forth in SEQ ID NO: 24.

In some embodiments, the nanobody may include a VHH chain having an amino acid sequence as set forth in SEQ ID NO: 6.

In some embodiments, the VHH chain may include a conservative substitution of less than or equal to 2 amino acids or less than or equal to 1 amino acid.

In some embodiments, the VHH chain may have an amino acid sequence with at least 90%, 92%, 94%, 96%, or 98% identity in the framework regions compared to the amino acid sequence as set forth in SEQ ID NO: 6. In some embodiments, the VHH chain may have an amino acid sequence having addition, deletion, and/or substitution of one or more amino acids in the framework regions compared to the amino acid sequence as set forth in SEQ ID NO: 6.

In some embodiments, the nanobody may be in an isolated state. In other embodiments, the nanobody may be recombinant.

Some embodiments of the present disclosure also provide a nanobody that binds specifically to GPRC5D. The nanobody may include a combination of three CDRs and the combination may include a CDR1, a CDR2, and a CDR3.

In some embodiments, an amino acid sequence of the CDR1 may be as set forth in SEQ ID NO: 25. An amino acid sequence of the CDR2 may be set forth in SEQ ID NO: 26. An amino acid sequence of the CDR3 may be set forth in SEQ ID NO: 27.

In some embodiments, the nanobody may include a VHH chain having an amino acid sequence as set forth in SEQ ID NO: 7.

In some embodiments, the VHH chain may include a conservative substitution of less than or equal to 2 amino acids or less than or equal to 1 amino acid.

In some embodiments, the VHH chain may have an amino acid sequence with at least 90%, 92%, 94%, 96%, or 98% identity in the framework regions compared to the amino acid sequence as set forth in SEQ ID NO: 7. In some embodiments, the VHH chain may have an amino acid sequence having addition, deletion, and/or substitution of one or more amino acids in the framework regions compared to the amino acid sequence as set forth in SEQ ID NO: 7.

In some embodiments, the nanobody may be in an isolated state. In other embodiments, the nanobody may be recombinant.

Some embodiments of the present disclosure also provide an anti-GPRC5D antibody. The antibody may include the nanobody.

In some embodiments, the antibody may be bispecific, bind to GPRC5D, and further bind to a second antigen.

Some embodiments of the present disclosure also provide an immunoconjugate. The immunoconjugate may include the nanobody. In some embodiments, the immunoconjugate may include an antibody that includes the nanobody.

Various methods can be used for preparing the immunoconjugate. For example, the nanobody or the antibody that includes the nanobody may be connected to a functional molecule directly or through a spacer of a suitable length. The connection may be chemical cross-linking or genetic engineering fusion expression, so as to obtain the immunoconjugate.

In some embodiments, the antibody may be conjugated to a cytotoxic agent, a radioisotope, a drug compound, or a peptide. The term “cytotoxic agent” refers to any agent that is detrimental to growth and proliferation of cells and acts to reduce, inhibit, or kill cells or malignant tumors. A non-limiting example of the functional molecule to which the antibody is conjugated may include, but is not limited to: an angiogenesis inhibitor (e.g., Thalidomide), an MAPK signaling pathway inhibitor (e.g., Metformin), an AKT signaling pathway inhibitor (e.g., Tanespimycin), a PI3K/m-TOR/AKT signaling pathway inhibitor (e.g., Bortezomib), a kinase inhibitor (e.g., Imatinib, Dovitinib), an HDAC inhibitor (e.g., Chidamide), a PARP inhibitor (e.g., PJ-34), a RNA polymerase inhibitor (e.g., Amanitin), a DNA damaging agent (e.g., Calicheamicin), a DNA alkylating agent (e.g., Melflufen), a DNA intercalation agent (e.g., Adriamycin), a DNA minor groove binder (e.g., Anthramycin), a ribosome inactivator (e.g., Gelonin), a microtubule stabilizer (e.g., Epothilone D), a microtubule destabilizer (e.g., Vincristine), a platinum compound (e.g., Carboplatin), a topoisomerase inhibitor (e.g., Topotecan), a strontium-89, a samarium-153, and a radium-223.

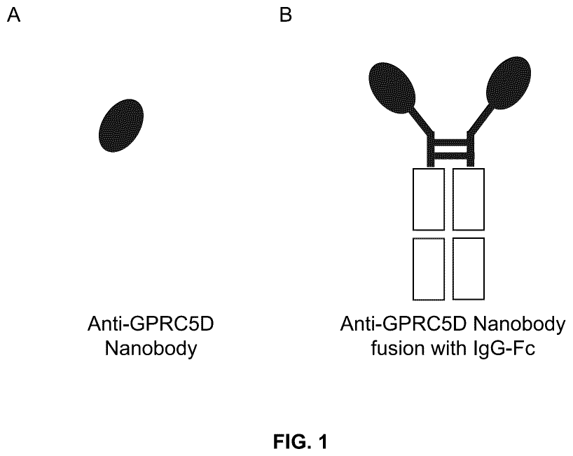

In some embodiments, the immunoconjugate may be a fusion protein constructed by fusion of the nanobody and a functional molecule, for example, the functional molecule may be used to prolong a half-life period of the antibody or have a binding effect on a specified effector cell. In some embodiments, the functional molecule used to prolong the half-life period of the antibody may include serum albumin or a fragment thereof, polyethylene glycol, etc. In some embodiments, the functional molecule having a binding effect to the effector cell may include a immunoglobulin Fc fragment. For example, the fusion protein may include the fused nanobody and the human immunoglobulin Fc fragment, which may prolong the half-life period of the fusion protein in a human body and enhance relevant effector functions (e.g., CDC activity, ADCC activity, and ADCP activity) mediated by Fc. In some embodiments, the fusion protein may be constructed by fusion of the nanobody and the IgG-Fc provided in the present disclosure (see FIG. 1).

In a specific embodiment, the fusion protein may include an amino acid sequence as set forth in SEQ ID NO: 8. Further, the amino acid sequence of the fusion protein may be as set forth in SEQ ID NO: 8.

In another specific embodiment, the fusion protein may include an amino acid sequence as set forth in SEQ ID NO: 9. Further, the amino acid sequence of the fusion protein may be as set forth in SEQ ID NO: 9.

In a further specific embodiment, the fusion protein may include an amino acid sequence as set forth in SEQ ID NO: 10. Further, the amino acid sequence of the fusion protein may be as set forth in SEQ ID NO: 10.

In still a further specific embodiment, the fusion protein may include an amino acid sequence as set forth in SEQ ID NO: 11. Further, the amino acid sequence of the fusion protein may be as set forth in SEQ ID NO: 11.

In still a further specific embodiment, the fusion protein may include an amino acid sequence as set forth in SEQ ID NO: 12. Further, the amino acid sequence of the fusion protein may be as set forth in SEQ ID NO: 12.

In some embodiments, the immunoconjugate may be a chimeric antigen receptor (CAR) expressed in an immune effector cell, for example, the immune effector cell may include a T lymphocyte, a NK cell, a NKT cell, or the like, or a combination thereof. While utilizing a killing effect of the nanobody or the antibody that include the nanobody provided in the present disclosure, the chimeric antigen receptor may also enable the immune effector cell to have a highly specific cytotoxic effect on a myeloma cell expressing GPRC5D.

Some embodiments of the present disclosure also provide an isolated nucleic acid capable of encoding the nanobody. Various methods can be used for isolating the nucleic acid, for example, the nucleic acid may be prepared by a recombinant DNA technology or may be isolated from other suitable sources.

Some embodiments of the present disclosure also provide a pharmaceutical composition. The pharmaceutical composition may include the nanobody.

Some embodiments of the present disclosure also provide a pharmaceutical composition. The pharmaceutical composition may include the anti-GPRC5D antibody, the immunoconjugate, or the isolated nucleic acid.

In some embodiments, the pharmaceutical composition may also include a pharmaceutically acceptable carrier such as a buffer, an antioxidant, a surfactant, a flavor correctant, a preservative, etc. A dosage form of the pharmaceutical composition may be selected according to a need, including but not limited to a tablet, an injection, a spray, etc.

Some embodiments of the present disclosure also provide use of the nanobody, the anti-GPRC5D antibody, the immunoconjugate, the isolated nucleic acid, or the pharmaceutical composition as a medicine.

Some embodiments of the present disclosure also provide use of the nanobody, the anti-GPRC5D antibody, the immunoconjugate, the isolated nucleic acid, or the pharmaceutical composition in treating a disorder or disease related to GPRC5D. In some embodiments, the disease may be MM.

Some embodiments of the present disclosure also provide a method for treating a patient suffering from a disease related to GPRC5D. The method may include administering a pharmacologically effective amount of the nanobody, the anti-GPRC5D antibody, immunoconjugate, the isolated nucleic acid, or the pharmaceutical composition to the patient. In some embodiments, the disease of the patient may be MM, such as early MM, intermediate MM, or advanced MM. Further, the method may treat the patient with cancer recurrence or cancer metastasis.

It is understood that the nanobody, the anti-GPRC5D antibody, the immunoconjugate, the isolated nucleic acid, or the pharmaceutical composition provided in the present disclosure may be administered alone as a medicament for treating the MM patient or may be administered in combination with other agents, which is not limited in the embodiment.

Some embodiments of the present disclosure also provide a method for detecting the presence of GPRC5D in a sample. The method may include: contacting the sample with the nanobody under conditions that allow for formation of a complex between the nanobody and GPRC5D; and determining whether the complex has been formed. In some embodiments, a process for determining whether the complex has been formed may include, but not be limited to, immunochromatography, immunoblotting (IBT), immunofluorescence, and chemiluminescence immunoassay (CLIA).

Some embodiments of the present disclosure also provide a recombinant host cell. The host cell may include a eukaryotic or prokaryotic host cell which produces the nanobody.

In some embodiments, the isolated nucleic acid may be inserted into a genome of the host cell using, for example, the recombinant DNA technology and the gene transfection manner well known in the art, so that a gene encoding the nanobody may be operatively connected to transcription and translation regulatory sequences, the transcription and translation of the gene encoding the nanobody may be achieved through an expression system of the host cell, and the nanobody provided by the present disclosure may be produced.

In some embodiments, the host cell may be preferably a eukaryotic cell, such as a rodent cell or a human-derived cell. The host cell suitable for expressing the nanobody provided herein may include, but is not limited to, NSO, CHO, CHOK1, perC.6, Tk-ts13, BHK, HEK293, COS-7, T98G, CV-1/EBNA, a L cell, C127, 3T3, HeLa, NS1, an Sp2/0 myeloma cell, etc.

Some embodiments of the present disclosure also provide a method for producing an anti-GPRC5D nanobody. The method may include: (i) culturing a host cell that expresses a nucleic acid encoding the nanobody, and (ii) purifying the anti-GPRC5D nanobody from a culture medium of the host cell.

The experimental methods in the following embodiments are conventional methods unless otherwise specified. The experimental materials used in the following embodiments are purchased from conventional biochemical reagent companies unless otherwise specified. The quantitative experiments in the following embodiments are all set up with three replicate experiments and the results are averaged.

### Examples

### Antigen Preparation

Two antigens for animal immunization were prepared: an HEK293 cell (GPRC5D-expressing HEK293 cell) line expressing GPRC5D and a purified GPRC5D membrane protein. Specifically, a gene (SEQ ID NO: 1) of human GPRC5D was connected to an expression vector pcDNA™3.4, the HEK293 cell (Invitrogen) was transfected and transiently overexpressed in the HEK293 cell using the recombinant expression vector pcDNA™3.4, and the HEK293 cell line expressing GPRC5D was obtained. Then, isolation and purification may be performed using detergent to obtain a Flag-tagged GPRC5D membrane protein (SEQ ID NO: 2) and the antigen may be stabilized by adding lipid stabilization.

Various methods can be used for antigen preparation is; see, for example: Daopeng Yuan, Zhongmin Liu, Jonas Kaindl, Shoji Maeda, Jiawei Zhao, Xiaoou Sun, Jun Xu, Peter Gmeiner, Hong-Wei Wang, Brian K. Kobilka. Activation of the 2B adrenergic receptor by the sedative sympatholytic dexmedetomidine. Nature chemical biology (2020) (https://doi.org/10.1038/s41589-020-0492-2).

### Animal Immunization and Phage Antibody Library Construction

After mixed and emulsified with adjuvant, the GPRC5D membrane protein and the HEK293 cell line overexpressing GPRC5D were used as antigens to immunize a healthy alpaca. Specifically, the alpaca was immunized six times at an interval of fourteen days and the blood of the alpaca was collected seven days after the sixth immunization.

Isolation of peripheral blood lymphocytes (PBMCs), extraction of total RNA, reverse transcription synthesis of cDNA, and PCR amplification and recovery of target VHH fragments were performed using the blood of the immunized alpaca.

The target VHH fragment and the phage vector pComb3XTT (QT078) were simultaneously digest using restriction endonuclease Sac1/Sal1 (NEB, R3156M/R3138M). A digested product was purified by agarose gel electrophoresis and ligated by T4 ligase (M0202M). A ligation product was converted into an electroporation-competent cell TG1 (Lucigen, 60502) and the TG1 cell was infected with helper phage M13K07 to generate a phage antibody library. It is understood that the target VHH fragment is inserted upstream of the gene III of the phage M13K07, so as to display the target VHH fragment on the coat protein G3P on the surface of the recombinant phage M13K07.

### Screening of Anti-GPRC5D Nanobody

The purified GPRC5D membrane protein was used as the antigen for three rounds of screening of the phage antibody library and a positive phage-displayed nanobody was isolated using a magnetic bead (Pierce, 88817). It is understood that the above operation exposes the phage-displayed nanobody to the antigen, so as to achieve the purpose of isolating and purifying the nanobody capable of specifically binding to the antigen. Specifically, the phage was blocked with bovine serum albumin (BSA) and the blocked phage was applied to the blocked Anti-Flag magnetic bead for negative selection. The unbound phage was mixed with the purified Flag-tagged GPRC5D membrane protein to form a mixture and the mixture was incubated with the blocked magnetic bead, from which the phage bound to the magnetic bead was recovered by a magnetic separator (positive selection). The bound phage was eluted with acidic buffer and amplified for subsequent two rounds of negative and positive selection.

The positive phage-displayed nanobody isolated after three rounds of screening was used as an candidate nanobody. The binding ability of the candidate nanobody to the GPRC5D membrane protein and the GPRC5D-expressing cell was evaluated by ELISA and sequenced by Sanger sequencing.

Various approaches can be used for the phage antibody library construction and the antibody screening; see, for example: Bazan J, Całkosiński I, Gamian A. Phage display—a powerful technique for immunotherapy: 1. Introduction and potential of therapeutic applications. Hum Vaccin Immunother. 2012 Dec. 1; 8 (12): 1817-28 (doi: 10.4161/hv.21703. Epub 2012 Aug. 21).

### Recombinant Antibody Constructed by Fusion of the Nanobody and IgG-Fc

Based on the sequencing result, the candidate nanobody was fused with IgG-Fc to construct the expression vector pcDNA™ 3.4 and the HEK293 cell was transfected using the recombinant expression vector pcDNA™ 3.4 and transiently overexpressed in the HEK293 cell. Cell culture supernatant was collected and isolated and purified using protein A resin to obtain the candidate recombinant antibody (also referred to as the fusion protein).

A purity analysis may be performed on the candidate recombinant antibody using high performance liquid chromatography-size exclusion chromatography (HPLC-SEC). Specifically, HPLC-SEC analysis was performed using TSKgel G3000SWxl SEC column (TOSOH). The condition is as follows: Phosphate buffer (pH7.4) was used as mobile phase and the flow rate is 1 ml/min. The volume injected is 20 ul with the concentration of 1 mg/ml. Each sample was run for 15 minutes and detected at 214 nm and 280 nm.

The purity analysis may be performed on the candidate recombinant antibody using sodium dodecyl sulfate-polyacrylamide gel electrophoresis (SDS-PAGE). Specifically, samples were mixed with loading buffer to 1 mg/ml, and DTT was used for reducing. Then samples were boiled at 100° C. for 5 min and 10 μg sample was loaded into the wells of SDS-PAGE gel (Bio Rad, 4568086), and run to separate at 130V. The gels were stained with Coomassie Brilliant Blue for 30 min, then discolored with water for 1 h, and scanned with imaging system (Bio rad, ChemiDoc™).

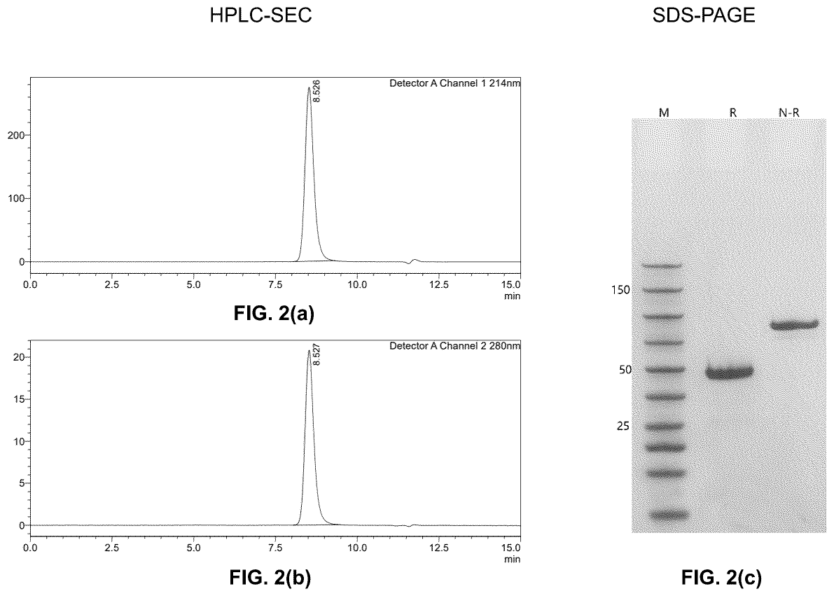

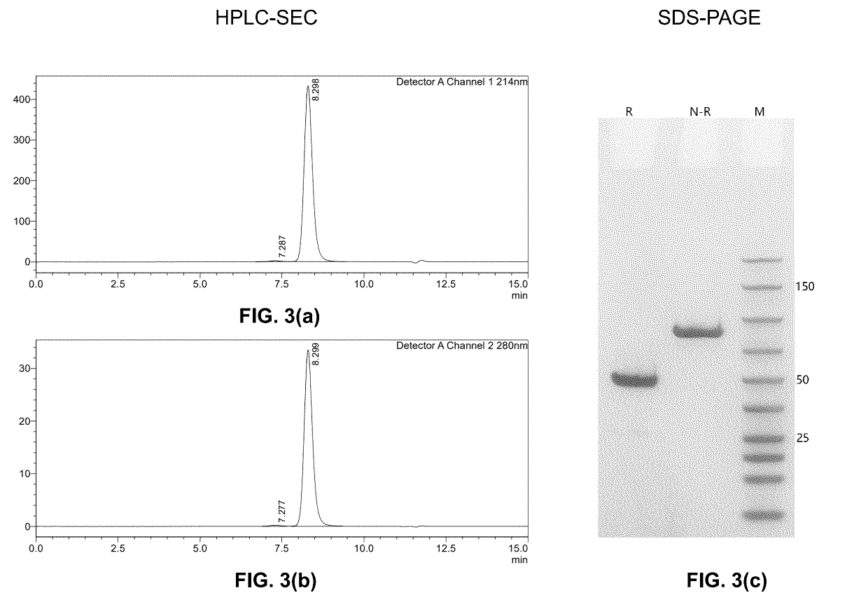

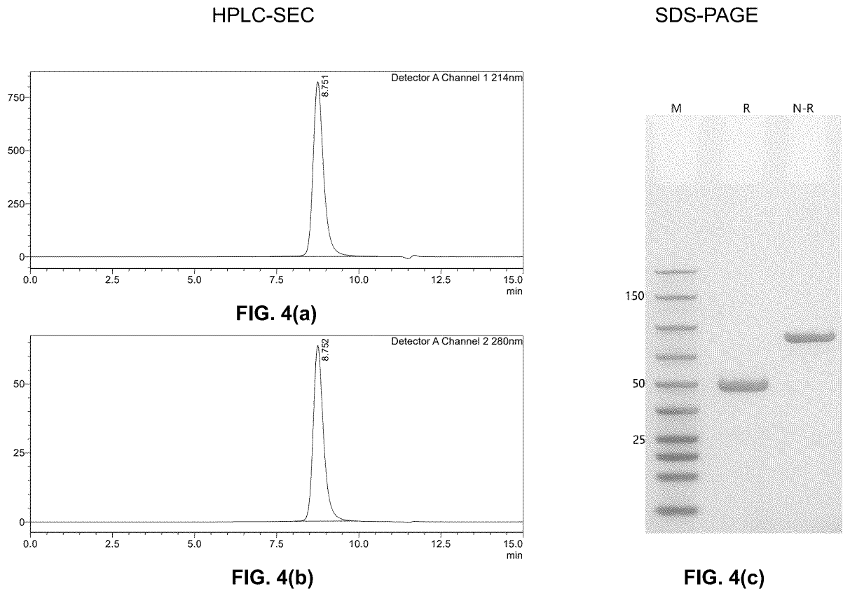

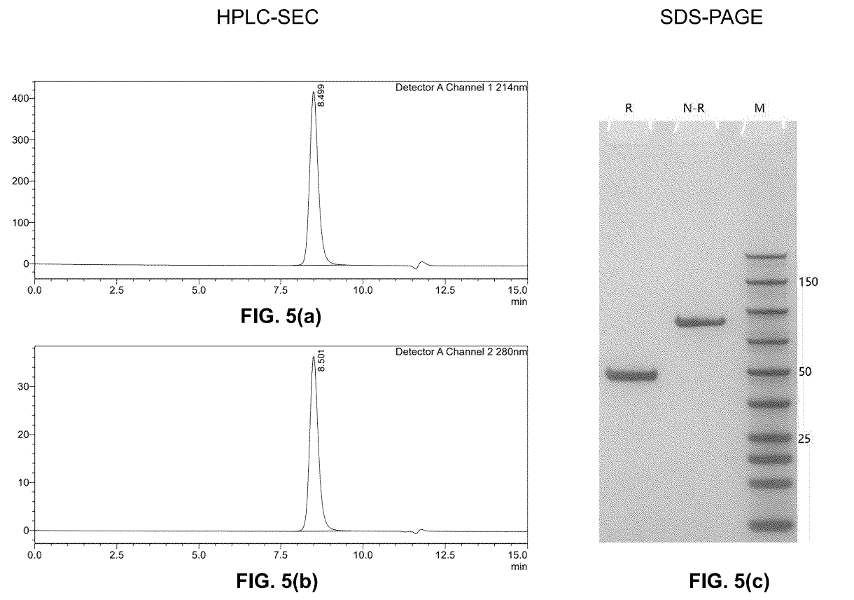

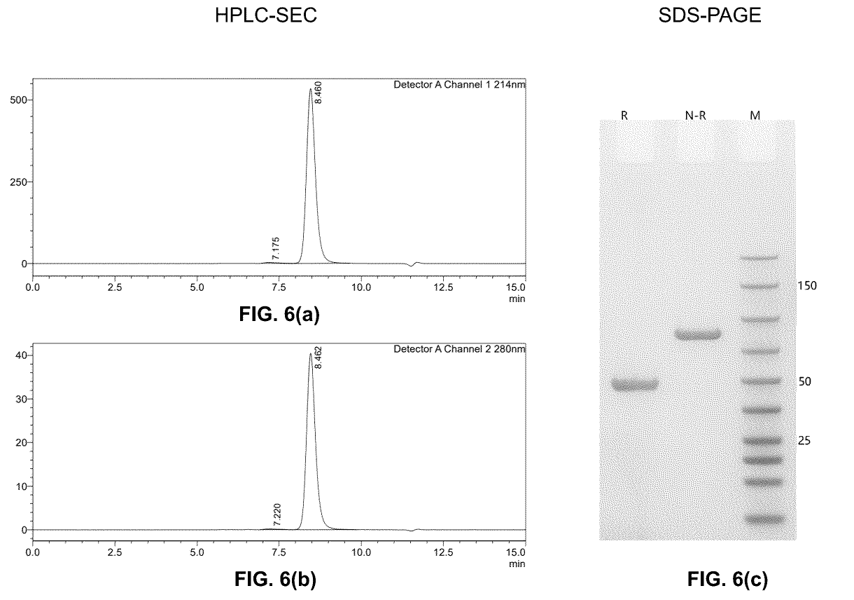

FIGS. 2(a)-2(c) show a purity identification result of a candidate recombinant antibody 1-F7-FC-fusion constructed by fusion of a candidate nanobody 1-F7 and the IgG-Fc according to some embodiments of the present disclosure. FIGS. 3(a)-3(c) show a purity identification result of a candidate recombinant antibody 1-E8-FC-fusion constructed by fusion of a candidate nanobody 1-E8 and the IgG-Fc according to some embodiments of the present disclosure. FIGS. 4(a)-4(c) show a purity identification result of a candidate recombinant antibody 1-D9-FC-fusion constructed by fusion of a candidate nanobody 1-D9 and the IgG-Fc according to some embodiments of the present disclosure. FIGS. 5(a)-5(c) show a purity identification result of a candidate recombinant antibody 1-C12-FC-fusion constructed by fusion of a candidate nanobody 1-C12 and the IgG-Fc according to some embodiments of the present disclosure. FIGS. 6(a)-6(c) show a purity identification result of a candidate recombinant antibody 1-G7-FC-fusion constructed by fusion of a candidate nanobody 1-G7 and the IgG-Fc according to some embodiments of the present disclosure. It is analyzed that, from the corresponding HPLC-SEC graph, there is basically no interference of impurity peaks and the purity is above 99%. It can be shown that, from the corresponding SDS-PAGE graph, the detected band size is correct and there are no other impurity bands, indicating that each candidate recombinant antibody after purification has a high purity and exists in the form of a monomer.

### Analysis of Antigen-Binding Ability of Nanobody and Recombinant Antibody Thereof

The affinity of the phage-displayed candidate nanobodies and the candidate recombinant antibodies with Fc fusions to the antigen was analyzed using the enzyme-linked immunosorbent assay (ELISA). Specifically, the phage-displayed candidate nanobodies and the candidate recombinant antibodies with Fc fusions were placed on plates for incubation. Detection was performed using a horseradish peroxidase (HRP)-conjugated secondary antibody. The purified GPRC5D membrane protein or the cell expressing human GPRC5D (e.g., a CHOK1 cell exogenously expressing GPRC5D, an MM1R cell endogenously expressing GPRC5D, an H929 cell endogenously expressing GPRC5D) were used as an antigen to similarly test the binding ability of the candidate nanobody and the candidate recombinant antibody. As a result, a target antibody with outstanding antigen binding ability and application potential can be screened out, such as the candidate nanobody 1-F7 (SEQ ID NO: 3), the candidate nanobody 1-E8 (SEQ ID NO: 4), the candidate nanobody 1-D9 (SEQ ID NO: 5), the candidate nanobody 1-C12 (SEQ ID NO: 6), the candidate nanobody 1-G7 (SEQ ID NO: 7), the candidate recombinant antibody 1-F7-FC-fusion (SEQ ID NO: 8), the candidate recombinant antibody 1-E8-FC-fusion (SEQ ID NO: 9), the candidate recombinant antibody 1-D9-FC-fusion (SEQ ID NO: 10), the candidate recombinant antibody 1-C12-FC-fusion (SEQ ID NO: 11), and the candidate recombinant antibody 1-G7-FC-fusion (SEQ ID NO: 12).

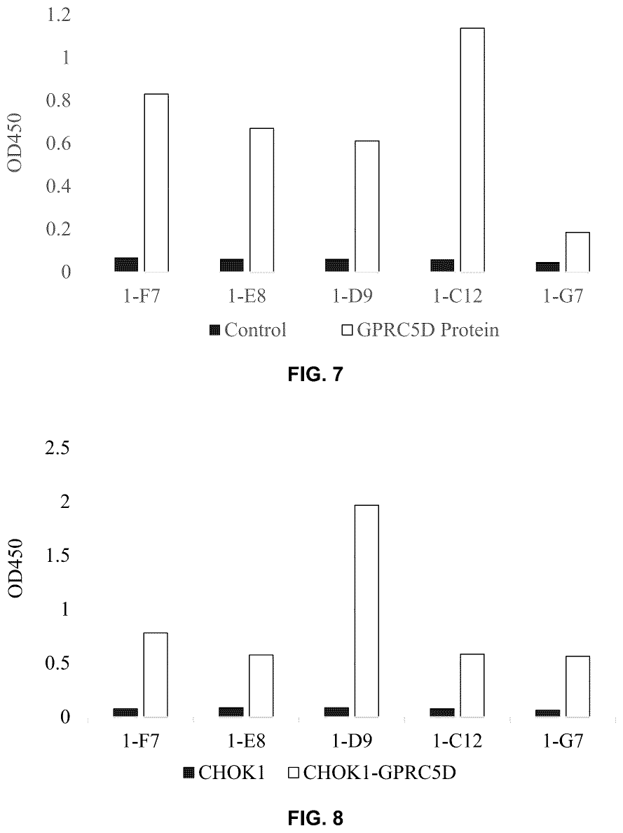

FIG. 7 shows a graph illustrating binding abilities of the phage-displayed candidate nanobodies to a purified GPRC5D protein detected by an ELISA according to some embodiments of the present disclosure. FIG. 8 shows a graph illustrating binding abilities of the phage-displayed candidate nanobodies to a CHOK1 cell transfected to express human GPRC5D (CHOK1-GPRC5D) detected by the ELISA according to some embodiments of the present disclosure. It can be seen from FIG. 7 and FIG. 8 that, compared with the control group, the candidate nanobody 1-F7, the candidate nanobody 1-E8, the candidate nanobody 1-D9, the candidate nanobody 1-C12, and the candidate nanobody 1-G7 have relatively good affinity and specificity for various antigens (e.g., a GPRC5D protein, a CHOK1 cell expressing human GPRC5D).

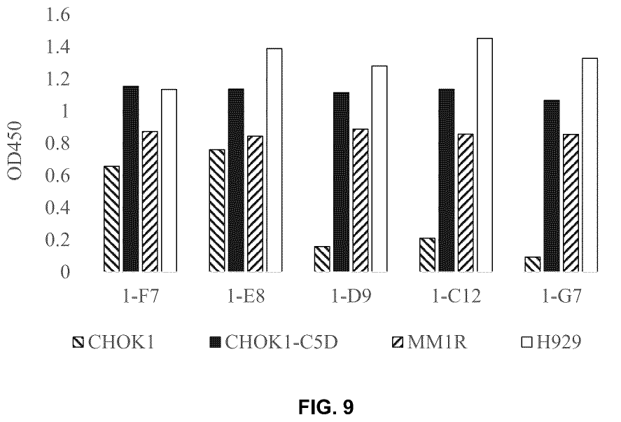

FIG. 9 shows a graph illustrating binding abilities of recombinant antibodies constructed by fusion of candidate nanobodies and Fc fragments to a cell expressing human GPRC5D detected by the ELISA according to some embodiments of the present disclosure. It can be seen from FIG. 9 that, compared with the control group, the candidate recombinant antibody 1-F7, the candidate recombinant antibody 1-E8, the candidate recombinant antibody 1-D9, the candidate recombinant antibody 1-C12, and the candidate recombinant antibody 1-G7 have relatively good affinity and specificity for various antigens (e.g., a CHOK1 cell overexpressing human GPRC5D (CHOK1-GPRC5D), an MM1R cell expressing human GPRC5D, an H929 cell expressing human GPRC5D).

The affinity of the phage-displayed candidate nanobodies and the candidate recombinant antibodies with Fc fusions to antigen was analyzed using the fluorescence-activated cell sorting (FACS). Specifically, the cell expressing human GPRC5D (e.g., a CHOK1 cell exogenously expressing human GPRC5D (CHOK1-GPRC5D), an MM1R cell endogenously expressing human GPRC5D, an H929 cell endogenously expressing human GPRC5D) was used as an antigen. After incubation with nanobodies in phage-displayed form and recombinant antibody form, a cell was obtained and blocked with bovine serum albumin (BSA). Detection was performed using an antigen presenting cell (APC)-conjugated anti-Flag fluorescent antibody (Biolegend, 637307).

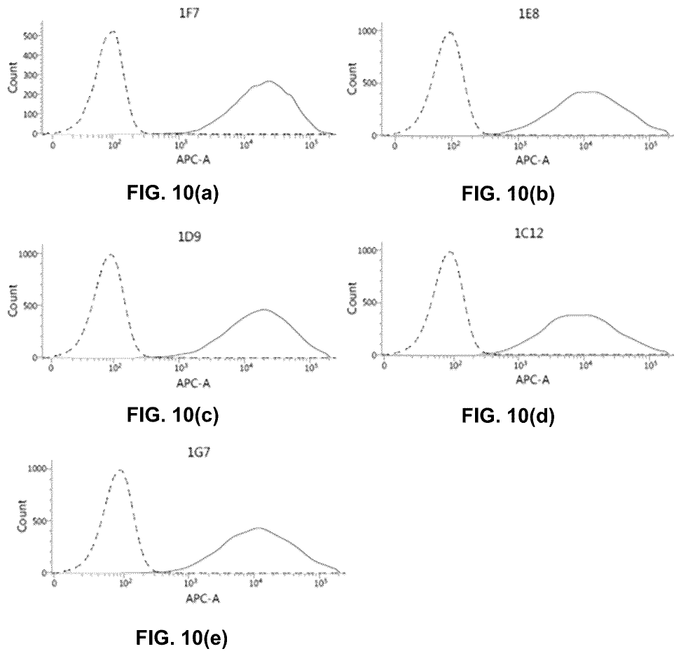

FIGS. 10(a)-10(e) show histograms illustrating binding abilities of the phage-displayed candidate nanobodies detected by a fluorescence-activated cell sorting (FACS) to a CHOK1-GPRC5D cell overexpressing human GPRC5D according to some embodiments of the present disclosure. The five candidate nanobodies showed binding to CHOK1-GPRC5D cells (solid line) compared to untransfected CHOK1 cells (spaced dot line).

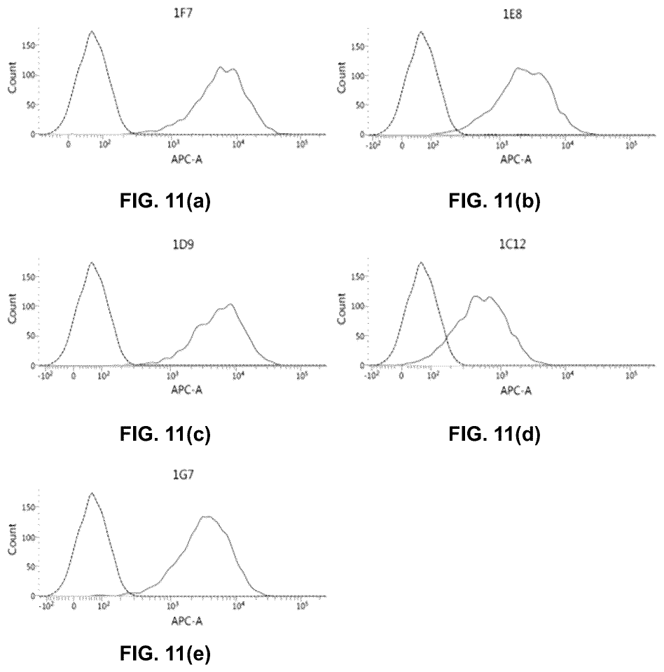

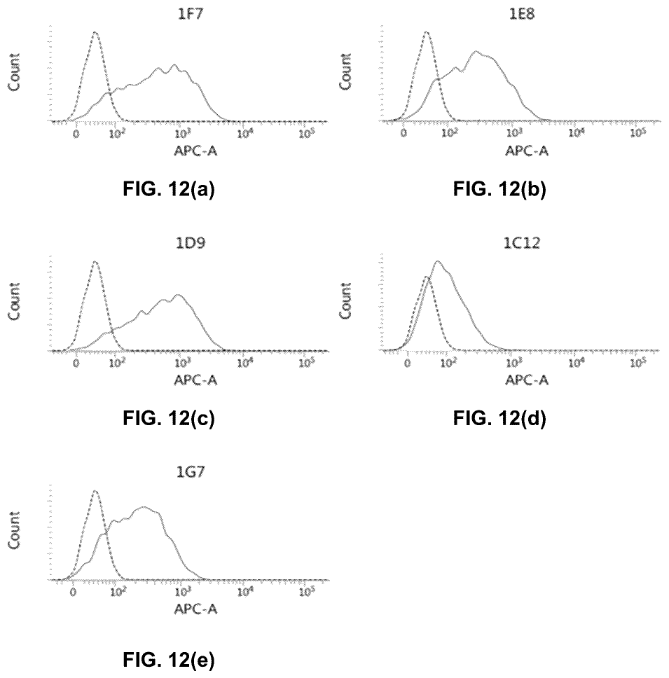

FIGS. 11(a)-11(e) show histograms illustrating binding abilities of the phage-displayed candidate nanobodies to an MM1R cell expressing human GPRC5D detected by the FACS according to some embodiments of the present disclosure. FIGS. 12(a) to 12(e) show histograms illustrating binding abilities of the phage-displayed candidate nanobodies to an H929 cell expressing human GPRC5D detected by the FACS according to some embodiments of the present disclosure. The five candidate nanobodies showed shifts in the histograms compared to the blank control (dash line), indicating their binding ability to the natural expressed GPRC5D protein on MM1R and H929 tumor cell lines (solid line).

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, some features, structures, or features in the present disclosure of one or more embodiments may be appropriately combined.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A nanobody that binds specifically to G protein-coupled receptor, class C, group 5, member D (GPRC5D), wherein the nanobody comprises a combination of three complementary determining regions (CDRs), and the combination is selected from the group consisting of:
(i) a CDR1 having an amino acid sequence as set forth in SEQ ID NO: 13, a CDR2 having an amino acid sequence as set forth in SEQ ID NO: 14, and a CDR3 having an amino acid sequence as set forth in SEQ ID NO: 15,
(ii) a CDR1 having an amino acid sequence as set forth in SEQ ID NO: 16, a CDR2 having an amino acid sequence as set forth in SEQ ID NO: 17, and a CDR3 having an amino acid sequence as set forth in SEQ ID NO: 18,
(iii) a CDR1 having an amino acid sequence as set forth in SEQ ID NO: 19, a CDR2 having an amino acid sequence as set forth in SEQ ID NO: 20, and a CDR3 having an amino acid sequence as set forth in SEQ ID NO: 21,
(iv) a CDR1 having an amino acid sequence as set forth in SEQ ID NO: 22, a CDR2 having an amino acid sequence as set forth in SEQ ID NO: 23, and a CDR3 having an amino acid sequence as set forth in SEQ ID NO: 24, and
(v) a CDR1 having an amino acid sequence as set forth in SEQ ID NO: 25, a CDR2 having an amino acid sequence as set forth in SEQ ID NO: 26, and a CDR3 having an amino acid sequence as set forth in SEQ ID NO: 27.

2. The nanobody of claim 1, which comprises a VHH chain having an amino acid sequence as set forth in SEQ ID Nos: 3, 4, 5, 6, or 7.

3. An anti-GPRC5D antibody comprising the nanobody of claim 1.

4. The antibody of claim 3, wherein the antibody is bispecific, binds to GPRC5D, and further binds to a second antigen.

5. An immunoconjugate comprising the nanobody of claim 1.

6. The immunoconjugate of claim 5, comprising an antibody that includes the nanobody.

7. The immunoconjugate of claim 6, wherein the antibody is conjugated to a cytotoxic agent, a radioisotope, a drug compound, or a peptide.

8. An isolated nucleic acid encoding the nanobody of claim 1.

9. A recombinant eukaryotic or prokaryotic host cell which produces the nanobody of claim 1.

10. A pharmaceutical composition comprising the nanobody of claim 1.

11. A pharmaceutical composition comprising the antibody of claim 3.

12-15. (canceled)

16. A method for treating a patient suffering from a disease related to GPRC5D, comprising:
administering a pharmacologically effective amount of the nanobody of claim 1 to the patient.

17. The method of claim 16, wherein the disease is multiple myeloma.

18. A method for detecting the presence of GPRC5D in a sample, the method comprising:
contacting the sample with a nanobody of claim 1 under conditions that allow for formation of a complex between the nanobody and GPRC5D; and
determining whether the complex has been formed.

19. A method for producing an anti-GPRC5D nanobody, the method comprising:
(i) culturing a host cell that expresses a nucleic acid encoding the nanobody of claim 1, and
(ii) purifying the anti-GPRC5D nanobody from a culture medium of the host cell.

20. A method for treating a patient suffering from multiple myeloma, comprising:
administering a pharmacologically effective amount of the antibody of claim 3 to the patient.

21. A method for treating a patient suffering from multiple myeloma, comprising:
administering a pharmacologically effective amount of the immunoconjugate of claim 5 to the patient.

22. A method for treating a patient suffering from multiple myeloma, comprising:
administering a pharmacologically effective amount of the isolated nucleic acid of claim 8 to the patient.

23. A method for treating a patient suffering from multiple myeloma, comprising:
administering a pharmacologically effective amount of the pharmaceutical composition of claim 10 to the patient.


SEQUENCE LISTING (27 sequences)

SEQ ID NO: 1 - protein, 374 aa, synthetic construct
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGVGGGGSDYKDDDDKDYKDDDDKDYKDDDDK

SEQ ID NO: 2 - protein, 24 aa, synthetic construct
DYKDDDDKDYKDDDDKDYKDDDDK

SEQ ID NO: 3 - protein, 115 aa, synthetic construct
QLQLVESGGGLVQPGGSLRLSCAASGSIFSVHDMAWYRQAPGKQRELVAAITSGGKANYPDSVKGRFTISRDNAKNTVYLEMNRLKPEDTAVYYCNKRWPVKYAWGQGTQVTVSS

SEQ ID NO: 4 - protein, 123 aa, synthetic construct
DVQLQESGGGLVQAGGSLRLSCAASGRAFSNYAMGWFRQAPGKEREFVAEASWSGRSTRYADSVKGRFTISRDNAKNTGYLQMSNLKPEDTAVYYCATSRTVVIGPGAKYDYWGQGTQVTVSS

SEQ ID NO: 5 - protein, 125 aa, synthetic construct
QVQLVESGGGSVQAGGSLRLSCAASERTFSNYAMGWFRQAPGKEREFVAAIAWSGEITNYADSVKGRFTVSRDIDKNTVYLQMNSLKPEDTAVYYCAGVRYRNYRTTRPTDFGSWGQGTQVTVSS

SEQ ID NO: 6 - protein, 125 aa, synthetic construct
EVQVVESGGGLVQAGGSLRVSCAASERTLSNYVMGWFRQAPGKSREFVAGIRWADGASYYADSVKGRFTISRDNAGNTVYLQMNSLKPEDTAVYYCTGGGIRTWSRKDPTSYRYWGQGTQVTVSS

SEQ ID NO: 7 - protein, 113 aa, synthetic construct
EVQLVESGGGLAQPGGSLRLSCAASGIISSAYIMGWYRQAPGKQRELVATINGGRLNYADSVKGRFTISSDNAKNTVYLQMNSLKPEDTAVYYCYAQRTNAYWGQGTQVTVSS

SEQ ID NO: 8 - protein, 364 aa, synthetic construct
QLQLVESGGGLVQPGGSLRLSCAASGSIFSVHDMAWYRQAPGKQRELVAAITSGGKANYPDSVKGRFTISRDNAKNTVYLEMNRLKPEDTAVYYCNKRWPVKYAWGQGTQVTVSSGGGGSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 372 aa, synthetic construct
DVQLQESGGGLVQAGGSLRLSCAASGRAFSNYAMGWFRQAPGKEREFVAEASWSGRSTRYADSVKGRFTISRDNAKNTGYLQMSNLKPEDTAVYYCATSRTVVIGPGAKYDYWGQGTQVTVSSGGGGSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 374 aa, synthetic construct
QVQLVESGGGSVQAGGSLRLSCAASERTFSNYAMGWFRQAPGKEREFVAAIAWSGEITNYADSVKGRFTVSRDIDKNTVYLQMNSLKPEDTAVYYCAGVRYRNYRTTRPTDFGSWGQGTQVTVSSGGGGSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 374 aa, synthetic construct
EVQVVESGGGLVQAGGSLRVSCAASERTLSNYVMGWFRQAPGKSREFVAGIRWADGASYYADSVKGRFTISRDNAGNTVYLQMNSLKPEDTAVYYCTGGGIRTWSRKDPTSYRYWGQGTQVTVSSGGGGSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 362 aa, synthetic construct
EVQLVESGGGLAQPGGSLRLSCAASGIISSAYIMGWYRQAPGKQRELVATINGGRLNYADSVKGRFTISSDNAKNTVYLQMNSLKPEDTAVYYCYAQRTNAYWGQGTQVTVSSGGGGSGGGGSGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 8 aa, synthetic construct
GSIFSVHD

SEQ ID NO: 14 - protein, 7 aa, synthetic construct
ITSGGKA

SEQ ID NO: 15 - protein, 9 aa, synthetic construct
NKRWPVKYA

SEQ ID NO: 16 - protein, 8 aa, synthetic construct
GRAFSNYA

SEQ ID NO: 17 - protein, 8 aa, synthetic construct
ASWSGRST

SEQ ID NO: 18 - protein, 16 aa, synthetic construct
ATSRTVVIGPGAKYDY

SEQ ID NO: 19 - protein, 8 aa, synthetic construct
ERTFSNYA

SEQ ID NO: 20 - protein, 8 aa, synthetic construct
IAWSGEIT

SEQ ID NO: 21 - protein, 18 aa, synthetic construct
AGVRYRNYRTTRPTDFGS

SEQ ID NO: 22 - protein, 8 aa, synthetic construct
ERTLSNYV

SEQ ID NO: 23 - protein, 8 aa, synthetic construct
IRWADGAS

SEQ ID NO: 24 - protein, 18 aa, synthetic construct
TGGGIRTWSRKDPTSYRY

SEQ ID NO: 25 - protein, 8 aa, synthetic construct
GIISSAYI

SEQ ID NO: 26 - protein, 6 aa, synthetic construct
INGGRL

SEQ ID NO: 27 - protein, 8 aa, synthetic construct
YAQRTNAY